(19) United States
(12) Patent Application Publication
(10) Pub. No.: US 2024/0331151 A1
(43) Pub. Date: Oct. 3, 2024

(54) METHOD AND APPARATUS FOR ASSESSMENT OF CHANGES IN QUANTITATIVE DIAGNOSTIC ASSISTANT INFORMATION FOR REGIONAL AIRWAY

(71) Applicants: CORELINE SOFT CO., LTD., Seoul (KR); The Asan Foundation, Seoul (KR); UNIV OF ULSAN FOUNDATION FOR INDUSTRY COOPERATION, Ulsan (KR)

(72) Inventors: Joon Beom SEO, Seoul (KR); Sang Min LEE, Seoul (KR); Hye Jeon HWANG, Seoul (KR); Seungbin BAE, Goyang-si, Gyeonggi-do (KR); Donghoon YU, Gimpo-si, Gyeonggi-do (KR); Jaeyoun YI, Seoul (KR)

(21) Appl. No.: 18/621,487

(22) Filed: Mar. 29, 2024

(30) Foreign Application Priority Data
Mar. 31, 2023 (KR) 10-2023-0043181

(51) Int. Cl. G06T 7/00; G06T 7/62; G06V 10/764; G16H 50/20

(52) U.S. Cl. CPC G06T 7/0012; G06T 7/62; G06V 10/764; G16H 50/20; G06T 2207/30101

(57) ABSTRACT

A method according to an embodiment of the present disclosure includes obtaining a first branch region of an anatomical tubular structure in a first medical image and quantitative information of the first branch region; identifying a second branch region matching the first branch region of the anatomical tubular structure in a second medical image obtained after the first medical image; obtaining quantitative information of the second branch region; and generating diagnostic assistant information based on the quantitative information of the first branch region and the quantitative information of the second branch region.

### CROSS-REFERENCE TO RELATED APPLICATION

This application claims priority from KR 10-2023-0043181 filed Mar. 31, 2023, which is incorporated herein by reference in its entirety.

### BACKGROUND

### 1. Technical Field

The present invention relates to technology for processing, analyzing, and visualizing medical images, and more particularly to technology for the assessment of changes in quantitative diagnostic assistant information in medical images to assist in the diagnosis of the chest.

### 2. Related Art

The content described in the present section simply provides background information for embodiments and does not constitute prior art.

Unlike X-ray imaging, computed tomography (CT) obtains cross-sectional images of the human body that is cut horizontally. Compared to simple X-ray imaging, CT has the advantage of being able to view structures and lesions more clearly because there are few overlaps between the structures. Furthermore, CT imaging is cheaper and requires a shorter examination time than magnetic resonance imaging (MRI), so that it is a basic examination method when lesions are suspected in most organs and diseases and the detailed examination thereof is required.

Meanwhile, COPD refers to various clinical symptoms in which the alveoli inside the lung are permanently damaged. COPD typically includes emphysema, in which the tissue that constitutes the alveoli is destroyed and cannot function normally. Recently, to quantitatively diagnose emphysema, there has emerged a technology that calculates the contrast of CT images using software and automatically/semi-automatically analyzes regions having a specific contrast value or less as an emphysema area.

Pulmonary Functional Test (PFT) is an evaluation index used in the diagnosis of chronic obstructive pulmonary disease (COPD). There is an attempt to use the results of segmentation of the airway within the lung region in order to analyze causes affecting PFT.

To assess the degree of progression of COPD or emphysema, it is necessary to compare and read medical images over several months or years.

In a conventional technology, the degree of progression of COPD or emphysema is assessed by comparing individually obtained quantitative assessment results or by visually comparing the latest image and the previous image through the naked eyes of a radiologist.

As a result, there are problems in that variations may occur depending on medical image modality, the change in the imaging method, the conditions of a patient during imaging, and the conditions of a radiologist during reading, and/or the like and missing information occurs.

### SUMMARY

The present invention has been conceived to overcome the above-described problems, and one of the objects of the present invention is to provide a consistent index for quantitative analysis using the registration between the latest image and the previous image.

One of the objects of the present invention is to propose an assessment index, the visualization of the index, and assessment criteria for the quantitative assessment of the degree of progression of COPD or emphysema.

One of the objects of the present invention is to provide technology for generating an index capable of quantitatively assessing the degree of progression of a specific disease based on a change in quantitative information over time in a branch region by using follow-up matching between branch regions of an anatomical tubular structure in medical images.

According to an aspect of the present invention, there may be provided a method of generating diagnostic assistant information using medical images, the method including: obtaining the first branch region of an anatomical tubular structure in a first medical image and the quantitative information of the first branch region; identifying a second branch region matching the first branch region of the anatomical tubular structure in a second medical image obtained after the first medical image; obtaining the quantitative information of the second branch region; and generating diagnostic assistant information based on the quantitative information of the first branch region and the quantitative information of the second branch region.

In the method according to the embodiment of the present invention, the generating may comprise generating follow-up information including the quantitative information of the first branch region and the quantitative information of the second branch region that match each other.

In the method according to the embodiment of the present invention, the generating may further comprise generating group correspondence information regarding which group is corresponding to at least one of the first branch region or the second branch region corresponding to the follow-up information from among a plurality of groups classified based on the quantitative information of the first branch region and the quantitative information of the second branch region included in the follow-up information.

In the method according to the embodiment of the present invention, the generating group correspondence information may comprise generating the group correspondence information by classifying each of the plurality of groups as at least one of a group in which the quantitative information of the second branch region has increased compared to the quantitative information of the first branch region, a group in which the quantitative information of the second branch region is the same as the quantitative information of the first branch region, or a group in which the quantitative information of the second branch region has decreased compared to the quantitative information of the first branch region.

In the method according to the embodiment of the present invention, the generating group correspondence information may comprise generating the group correspondence information by further classifying each of the plurality of groups as at least one of a group to which a new third branch region that does not match the first branch region corresponds among branch regions found in the second medical image, or a group to which a fourth branch region that does not match the branch regions in the second medical image corresponds out of the first branch region.

The method according to the embodiment of the present invention may further comprise visualizing the diagnostic assistant information by visualizing the follow-up information together with a location of at least one of the first branch region or the second branch region corresponding to the follow-up information.

The method according to the embodiment of the present invention may further comprise visualizing the diagnostic assistant information by visualizing group correspondence information regarding which group is corresponding to at least one of the first branch region or the second branch region corresponding to the follow-up information.

In the method according to the embodiment of the present invention, the visualizing the diagnostic assistant information may comprise visualizing at least one of the first branch region or the second branch region using a visualization element designated for each group.

In the method according to the embodiment of the present invention, the quantitative information of at least one of the first branch region or the second branch region may comprise at least one of a volume, diameter, or sectional area of the branch region.

In the method according to the embodiment of the present invention, the quantitative information of at least one of the first branch region or the second branch region may comprise at least one of a thickness of a wall of a tubular structure in the branch region, a sectional area of the wall of the tubular structure in the branch region, a diameter of a lumen of the tubular structure in the branch region, a sectional area of the lumen of the tubular structure in the branch region, a normalized wall thickness of the tubular structure in the branch region, or a tapering ratio of the tubular structure in the branch region.

In the method according to the embodiment of the present invention, the generating the group correspondence information may comprise generating the group correspondence information by using a plurality of groups for each of which a change between the quantitative information of the first branch region and the quantitative information of the second branch region included in the follow-up information is classified by applying a predetermined threshold.

In the method according to the embodiment of the present invention, the anatomical tubular structure is at least one of an airway, an aorta, or a blood vessel.

According to an embodiment of the present invention, there may be provided an apparatus for generating diagnostic assistant information using medical images, the apparatus comprising: memory configured to store one or more instructions; and a processor configured to execute at least one of the instructions; wherein the processor is further configured to, in accordance with at least one of the instructions: obtain a first branch region of an anatomical tubular structure in a first medical image and quantitative information of the first branch region; identify a second branch region matching the first branch region of the anatomical tubular structure in a second medical image obtained after the first medical image; obtain quantitative information of the second branch region; and generate diagnostic assistant information based on the quantitative information of the first branch region and the quantitative information of the second branch region.

The processor may be further configured to, when generating the diagnostic assistant information, generate follow-up information including the quantitative information of the first branch region and the quantitative information of the second branch region that match each other.

The processor may be further configured to, when generating the diagnostic assistant information, generate group correspondence information regarding which group is corresponding to at least one of the first branch region or the second branch region corresponding to the follow-up information from among a plurality of groups classified based on the quantitative information of the first branch region and the quantitative information of the second branch region included in the follow-up information.

The processor may be further configured to, when generating the group correspondence information, generate the group correspondence information by classifying each of the plurality of groups as at least one of a group in which the quantitative information of the second branch region has increased compared to the quantitative information of the first branch region, a group in which the quantitative information of the second branch region is the same as the quantitative information of the first branch region, or a group in which the quantitative information of the second branch region has decreased compared to the quantitative information of the first branch region.

The processor may be further configured to, when generating the group correspondence information, generate the group correspondence information by further classifying each of the plurality of groups as at least one of a group to which a new third branch region that does not match the first branch region corresponds among branch regions found in the second medical image, or a group to which a fourth branch region that does not match the branch regions in the second medical image corresponds out of the first branch region.

The processor may be further configured to, in accordance with at least one of the instructions, visualize the diagnostic assistant information by visualizing the follow-up information together with a location of at least one of the first branch region or the second branch region corresponding to the follow-up information.

The processor may be further configured to, in accordance with at least one of the instructions, visualize the diagnostic assistant information by visualizing group correspondence information regarding which group is corresponding to at least one of the first branch region or the second branch region corresponding to the follow-up information.

The processor may be further configured to, when visualizing the diagnostic assistant information, visualize at least one of the first branch region or the second branch region using a visualization element designated for each group.

### BRIEF DESCRIPTION OF THE DRAWINGS

FIG. 1 is an operational flowchart showing a method of generating diagnostic assistant information using medical images according to an embodiment of the present invention;

FIG. 2 is an operational flowchart showing part of the process of FIG. 1 in a method according to an embodiment of the present invention in detail;

FIG. 3 is an operational flowchart showing a method of generating and visualizing diagnostic assistant information using medical images according to another embodiment of the present invention;

FIG. 4 is an operational flowchart showing a method of generating and visualizing diagnostic assistant information using medical images according to another embodiment of the present invention;

FIG. 5 is an operational flowchart showing a method of generating and visualizing diagnostic assistant information using medical images according to another embodiment of the present invention;

FIG. 6 is a conceptual diagram showing a process of quantifying the airway wall thickness as the quantitative information of an anatomical tubular structure according to an embodiment of the present invention;

FIG. 7 is a conceptual diagram showing an example of a table that provides the quantitative information of the wall and lumen of the airway for each branch region as the quantitative information of an anatomical tubular structure according to an embodiment of the present invention;

FIG. 8 is a conceptual diagram showing an embodiment of visualizing the follow-up quantitative information of an anatomical tubular structure according to an embodiment of the present invention;

FIG. 9 is a conceptual diagram showing a process of generating the quantitative information of an anatomical tubular structure according to an embodiment of the present invention;

FIG. 10 is an operational flowchart showing part of a process of generating the follow-up quantitative information of an anatomical tubular structure according to an embodiment of the present invention;

FIG. 11 is an operational flowchart showing another part of the process of generating the follow-up quantitative information of an anatomical tubular structure according to an embodiment of the present invention;

FIG. 12 is a conceptual diagram showing a process of the registration and matching of an anatomical tubular structure according to an embodiment of the present invention; and

FIG. 13 is a conceptual diagram showing an example of an apparatus or a generalized computing system for generating and visualizing diagnostic assistant information for the follow-up analysis of a specific disease.

### DETAILED DESCRIPTION

Other objects and features of the present invention in addition to the above-described objects will be apparent from the following description of embodiments to be given with reference to the accompanying drawings.

The embodiments of the present invention will be described in detail below with reference to the accompanying drawings. In the following description, when it is determined that a detailed description of a known component or function may unnecessarily make the gist of the present invention obscure, it will be omitted.

Relational terms such as first, second, A, B, and the like may be used for describing various elements, but the elements should not be limited by the terms. These terms are only used to distinguish one element from another. For example, a first component may be named a second component without departing from the scope of the present disclosure, and the second component may also be similarly named the first component. The term “and/or” means any one or a combination of a plurality of related and described items.

In embodiments of the present disclosure, “at least one of A and B” may mean “at least one of A or B” or “at least one of combinations of one or more of A and B.” Additionally, in embodiments of the present disclosure, “one or more of A and B” may mean “one or more of A or B” or “one or more of combinations of one or more of A and B.”

When it is mentioned that a certain component is “coupled with” or “connected with” another component, it should be understood that the certain component is directly “coupled with” or “connected with” to the other component or a further component may be disposed therebetween. In contrast, when it is mentioned that a certain component is “directly coupled with” or “directly connected with” another component, it will be understood that a further component is not disposed therebetween.

The terms used in the present disclosure are only used to describe specific exemplary embodiments, and are not intended to limit the present disclosure. The singular expression includes the plural expression unless the context clearly dictates otherwise. In the present disclosure, terms such as ‘comprise’ or ‘have’ are intended to designate that a feature, number, step, operation, component, part, or combination thereof described in the specification exists, but it should be understood that the terms do not preclude existence or addition of one or more features, numbers, steps, operations, components, parts, or combinations thereof.

Unless otherwise defined, all terms (including technical and scientific terms) used herein have the same meaning as commonly understood by one of ordinary skill in the art to which this disclosure belongs. Terms that are generally used and have been in dictionaries should be construed as having meanings matched with contextual meanings in the art. In this description, unless defined clearly, terms are not necessarily construed as having formal meanings.

Meanwhile, even if a technology is known prior to the filing date of the present disclosure, it may be included as part of the configuration of the present disclosure when necessary, and will be described herein without obscuring the spirit of the present disclosure. However, in describing the configuration of the present disclosure, a detailed description on matters that can be clearly understood by those skilled in the art as a known technology prior to the filing date of the present disclosure may obscure the purpose of the present disclosure, so excessively detailed description on the known technology will be omitted.

For example, technologies known before the application of the present invention may be used for a technology for the detection, segmentation, and classification of a specific organ of the human body and a sub-region of an organ by processing medical images, a technology for generating quantified information by measuring a segmented organ or finding region, etc. At least some of these known technologies may be applied as elemental technologies required for practicing the present invention.

In related prior literature, artificial neural networks are used to detect and classify lesion candidates and generate findings. Each of the findings includes diagnostic assistant information, and the diagnostic assistant information includes quantitative measurements such as the probability that each finding is actually a lesion, the confidence of the finding, the degree of malignancy, and the size and volume of lesion candidates corresponding to the finding.

In medical image diagnosis assistance using artificial neural networks, each finding needs to be quantified in the form of probability or confidence and included as diagnostic assistant information, and all findings may not be provided to a user. Accordingly, in general, findings are filtered by applying a specific threshold thereto, and only the findings obtained through the filtering are provided to the user. In a workflow in which a user, who is a radiologist, reads medical images and then generates clinical findings and a clinician analyzes the findings and then generates diagnosis results, an artificial neural network or automation program may assist in at least part of the reading process and finding generation process of the radiologist and the diagnosis process of the clinician.

However, the purpose of the present invention is not to claim rights to these known technologies, and the content of the known technologies may be included as part of the present invention within the range that does not depart from the spirit of the present invention.

Embodiments of the present invention will be described in detail below with reference to the accompanying drawings. In order to facilitate overall understanding in the following description of the present invention, the same reference numerals will be used for the same components throughout the drawings, and redundant descriptions thereof will be omitted.

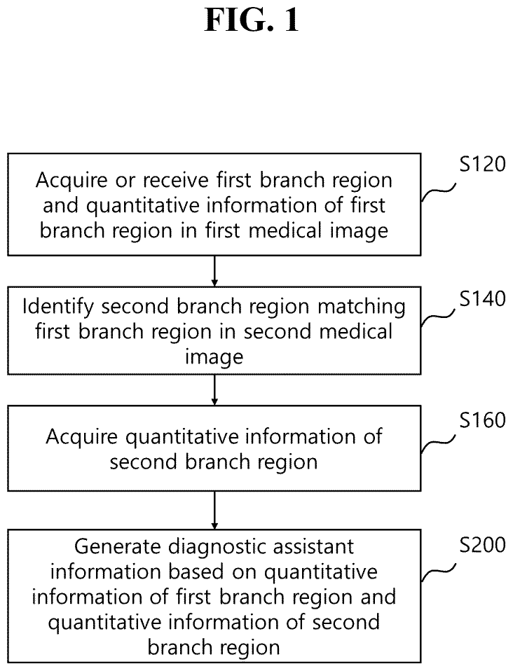

FIG. 1 is an operational flowchart showing a method of generating diagnostic assistant information using medical images according to an embodiment of the present invention.

Referring to FIG. 1, the method of generating diagnostic assistant information using medical images according to an embodiment of the present invention may include step S120 of obtaining the first branch region of an anatomical tubular structure in a first medical image and the quantitative information of the first branch region; step S140 of identifying a second branch region matching the first branch region of the anatomical tubular structure in a second medical image obtained after the first medical image; step S160 of obtaining the quantitative information of the second branch region; and step S200 of generating diagnostic assistant information based on the quantitative information of the first branch region and the quantitative information of the second branch region.

In the method of generating diagnostic assistant information using medical images according to an embodiment of the present invention, the anatomical tubular structure may include the airway, the aorta, and/or a blood vessel, and so on.

When branching occurs in the anatomical tubular structure, individual regions before and after branching may be identified as separate branch regions. Branch regions may each be specified by a branch level and hierarchical information (e.g., a tree structure) up to the current branch level.

In a level-0 branch region that circulates like the aorta, regions before and after the occurrence of another branching may be treated as separate branch regions.

The branch regions matching each other between the first medical image and second medical image may refer to a pair of branch regions for which a branch level and hierarchical information up to the current branch level are the same in the anatomical tubular structure.

To implement the present invention, neither the proposal of a new clinically useful branch region, nor a new method of segmenting branch regions, nor a new method of determining the branch level of a branch region is necessarily required. Furthermore, to implement the present invention, a new method for quantifying branch regions is not necessarily required.

A new medical finding region or new size information is not necessarily required to implement the present invention. The configurational feature of the present invention is to match branch regions, known in related art, in the first medical image, which is a baseline image, and the second medical image, which is a follow-up image, by using registration or matching and then generate or obtain clinically meaningful follow-up information by using a change in quantitative information between matching branch regions obtained by a known method.

An embodiment of the present invention may propose a representation format for generating changes in quantitative information between matching branch regions as clinically meaningful follow-up information. Furthermore, an embodiment of the present invention may obtain a means of quantitative assessment of the degree of progression of a specific disease by quantitatively classifying a combination of the pieces of quantitative information between matching branch regions or a change in quantitative information between matching branch regions.

The matching branch regions may be specified and matched with each other by a branch level and hierarchical information (e.g., a tree structure) up to the current branch level.

An embodiment of the present invention may provide a means for visualizing the means of quantitative assessment of the degree of progression of a specific disease obtained by quantitatively classifying a combination of the pieces of quantitative information between matching branch regions or a change in quantitative information between matching branch regions.

In an embodiment of the present invention, the quantitative information between matching branch regions may be represented by an ordered pair such as (the quantitative information of the first branch region, the quantitative information of the second branch region). In an alternative embodiment of the present invention, the change in quantitative information between branch regions may be represented by the difference or ratio in quantitative information.

In an embodiment of the present invention, the quantitative information between branch regions may be represented by values, or may be represented by size sections or groups classified by the values of the quantitative information.

The branch region may be obtained through thresholding, detection, segmentation, and/or classification in the medical image.

The medical image may be a CT image, a magnetic resonance (MR) image, an X-ray image, or the like, and the branch region may include a medical branch region that can be obtained from a known modality.

Alternative examples of the branch region that can be extracted by considering intensity and shape in the medical image may include the airway, the aorta, or a blood vessel, or the like.

The branch region may be obtained through detection, segmentation, and/or classification based on the results of thresholding of intensity values.

Follow-up information regarding matching branch regions may be provided for a pair of branch regions for which matching has been successfully performed. According to an alternative embodiment of the present invention, when matching fails, information regarding branch regions for which matching has failed may be included in follow-up information and be then provided additionally.

In the method of generating diagnostic assistant information using medical images according to an embodiment of the present invention, the quantitative information of the first branch region or the second branch region may include the volume, diameter, or sectional area of the branch region, or the like.

According to an alternative embodiment of the present invention, the quantitative information of the branch region may include the length of the branch region, the length of the perimeter of the branch region, the length of the long axis of the section, the length of the short axis of the section, the product of the lengths of the long and short axes of the section, and/or the volume of the branch region.

For convenience of description, the length of the long axis may be understood as referring to various measurement methods that can be used for the quantitative analysis of a specific branch or finding region in a comprehensive manner. The quantitative measurement methods for a branch or finding region include methods of measuring, e.g., the length of the long axis, the length of the short axis, the effective diameter, and/or the average length.

In the method of generating diagnostic assistant information using medical images according to an embodiment of the present invention, the quantitative information of the first branch region or the second branch region may include the thickness of the wall of the tubular structure in the branch region, the sectional area of the wall of the tubular structure in the branch region, the diameter of the lumen of the tubular structure in the branch region, the sectional area of the lumen of the tubular structure in the branch region, the normalized/standardized wall thickness of the tubular structure in the branch region (e.g. AWT-Pi10 in the case of the airway wall), and/or the tapering ratio of the tubular structure in the branch region. In this case, the tapering ratio may refer to the rate at which the length of the perimeter, the diameter, the sectional area, or the like decreases or increases as the branch region is farther away from a higher level based on a branch point. Depending on a clinical need for diagnosis, either decrease or increase may be treated as more important.

The quantitative information of the first branch region and the quantitative information of the second branch region that match each other may be calculated by the same method for the purpose of comparison therebetween.

State information to be obtained using quantitative information for the purpose of diagnosis of a lesion or disease for an anatomical tubular structure may include the occlusion of the tubular structure, the distortion of the tubular structure, the separation of the wall and lumen of the tubular structure, and the rupture or loss of the tubular structure. For example, pulmonary embolism is an example of a disease related to the occlusion of the pulmonary artery. To analyze a specific disease using the quantitative information of the anatomical tubular structure, the quantitative information of a finding region in addition to the quantitative information of a branch region may also be taken into consideration.

The finding region may be a lesion or tumor, or may refer to an anatomical structure having a predetermined special shape or structure. Furthermore, the finding region may refer to an anatomical region identified through thresholding, detection, segmentation, and/or classification according to special conditions.

Alternative examples of the finding region that can be extracted by considering intensity and shape in a medical image may include fat, blood, thrombus, etc.

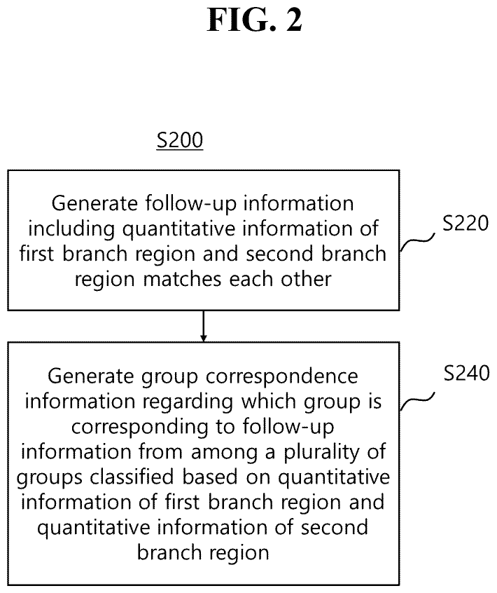

FIG. 2 is an operational flowchart showing part of the process of FIG. 1 in a method according to an embodiment of the present invention in detail.

Referring to FIG. 2, step S200 of generating diagnostic assistant information may include step S220 of generating follow-up information including the quantitative information of the first branch region and the quantitative information of the second branch region that match each other.

Step S200 of generating diagnostic assistant information may further include step S240 of generating group correspondence information regarding which group is corresponding to at least one of the first branch region and/or the second branch region corresponding to the follow-up information from among a plurality of groups classified based on the quantitative information of the first branch region and the quantitative information of the second branch region included in the follow-up information.

In step S240 of generating group correspondence information, the group corresponding information may be generated by classifying each of the plurality of groups as at least one of a group in which the quantitative information of the second branch region has increased compared to the quantitative information of the first branch region, a group in which the quantitative information of the second branch region is the same as the quantitative information of the first branch region, and/or a group in which the quantitative information of the second branch region has decreased compared to the quantitative information of the first branch region.

In this case, the group in which the quantitative information of the second branch region is the same as the quantitative information of the first branch region may correspond to a case where the change in quantitative information is less than a predetermined threshold.

The group in which the quantitative information of the second branch region has increased compared to the quantitative information of the first branch region may correspond to a case where the increase in quantitative information is equal to or greater than a predetermined threshold.

The group in which the quantitative information of the second branch region has decreased compared to the quantitative information of the first branch region may correspond to a case where the decrease in quantitative information is equal to or greater than a predetermined threshold.

In step S240 of generating group correspondence information, the group corresponding information may be generated by further classifying each of the plurality of groups as at least one of a group to which a new third branch region that does not match the first branch region corresponds among the branch regions found in the second medical image, and/or a group to which a fourth branch region that does not match the branch regions in the second medical image corresponds out of the first branch region.

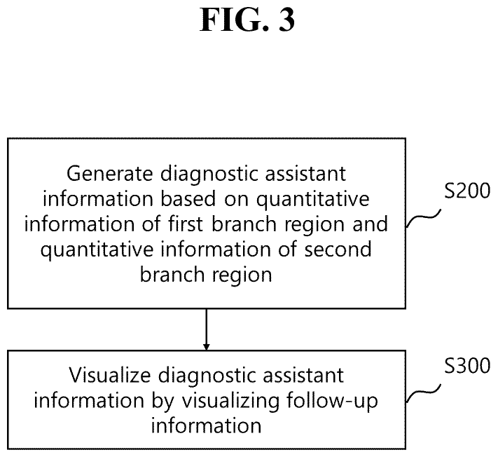

FIG. 3 is an operational flowchart showing a method of generating and visualizing diagnostic assistant information using medical images according to another embodiment of the present invention.

Referring to FIG. 3, the method of generating diagnostic assistant information using medical images according to an embodiment of the present invention may further include step S300 of visualizing diagnostic assistant information by visualizing the follow-up information generated in step S200.

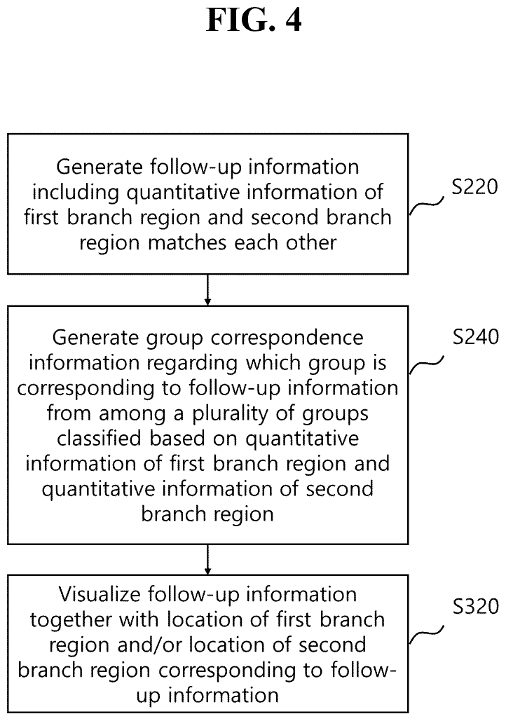

FIG. 4 is an operational flowchart showing a method of generating and visualizing diagnostic assistant information using medical images according to another embodiment of the present invention.

Referring to FIG. 4, the method of generating diagnostic assistant information using medical images according to an embodiment of the present invention may further include, as part of step S300, step S320 of visualizing the diagnostic assistant information by visualizing the follow-up information together with the location of at least one of the first branch region and/or the second branch region corresponding to the follow-up information.

In this case, when the first medical image is selected as a reference for a visualization process, the location of the first branch region may be visualized. When the second medical image is selected, the location of the second branch region may be visualized.

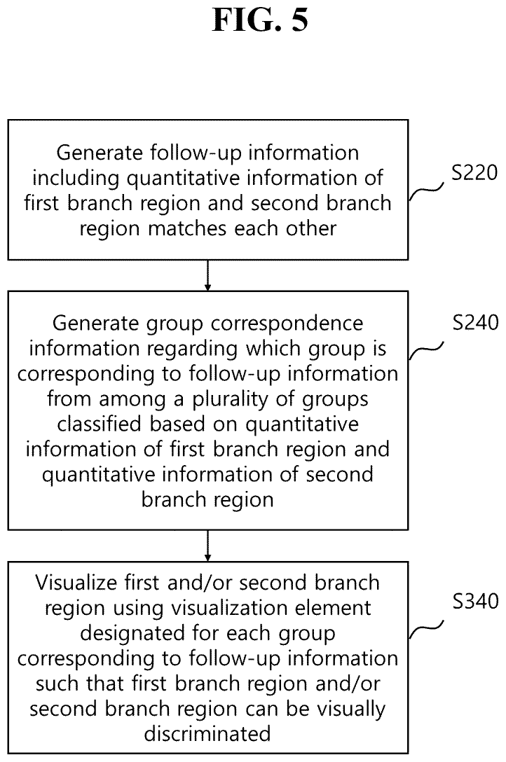

FIG. 5 is an operational flowchart showing a method of generating and visualizing diagnostic assistant information using medical images according to another embodiment of the present invention.

Referring to FIG. 5, the method of generating diagnostic assistant information using medical images according to an embodiment of the present invention may further include, as part of step S300, step S340 of visualizing diagnostic assistant information by visualizing group correspondence information regarding which group is corresponding to at least one of the first branch region and/or the second branch region corresponding to the follow-up information.

In steps S300 and S340 of visualizing diagnostic assistant information, at least one of the first branch region and/or the second branch region may be visualized using a visualization element designated for each group.

In step S240 of generating group correspondence information, the group correspondence information may be generated using a plurality of groups for each of which a change between the quantitative information of the first branch region and the quantitative information of the second branch region included in the follow-up information is classified by applying a predetermined threshold.

In this case, a section or group may be set by classifying the quantitative information of the first branch region in the baseline image and the quantitative information of the second branch region in the follow-up image using a threshold value.

According to an alternative embodiment, a section or group may be set by classifying only the change amount/change rate using a threshold regardless of the absolute values of the quantitative information of the first branch region in the baseline image and the quantitative information of the second branch region in the follow-up image.

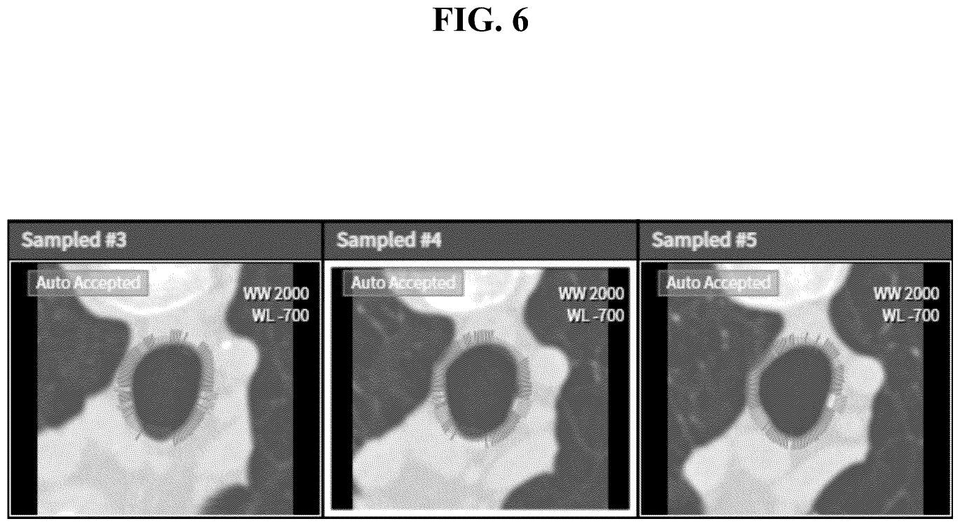

FIG. 6 is a conceptual diagram showing a process of quantifying the airway wall thickness as the quantitative information of an anatomical tubular structure according to an embodiment of the present invention.

Referring to FIG. 6, the airway walls of different segments within a branch region may be segmented, and the thicknesses of the airway walls may be quantified.

In an alternative embodiment of the invention, individual airway walls may be segmented for different branch regions, and the thicknesses of the airway walls may be quantified.

There may be provided a menu through which a medical professional can accept airway wall segmentation results for each segment or branch region and the resulting quantification results of the airway wall thicknesses. The airway wall segmentation results and the quantification results of the airway wall thicknesses may be automatically accepted by an engine that has been trained on a predetermined function.

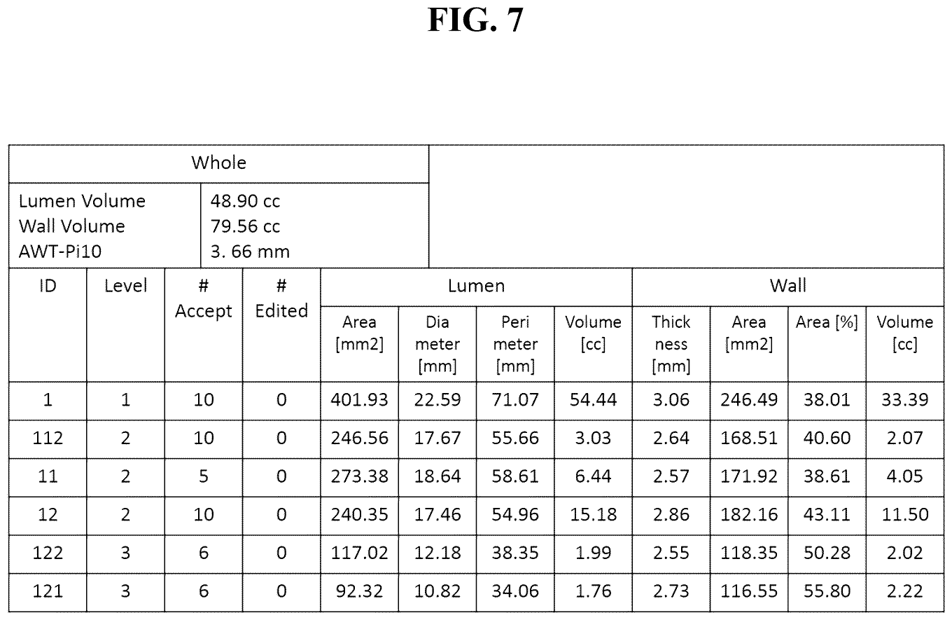

FIG. 7 is a conceptual diagram showing an example of a table that provides the quantitative information of the wall and lumen of the airway for each branch region as the quantitative information of an anatomical tubular structure according to an embodiment of the present invention.

Referring to FIG. 7, the sectional area, diameter, peripheral length, and volume of the lumen may be quantified for each branch area.

Furthermore, the thickness, sectional area, area % (the ratio of the sectional area of the wall to the total sectional area of the lumen and wall), and volume of the wall of the branch region may be quantified for each branch region.

In FIG. 7, each airway region may be specified and identified by an ID and a branch level.

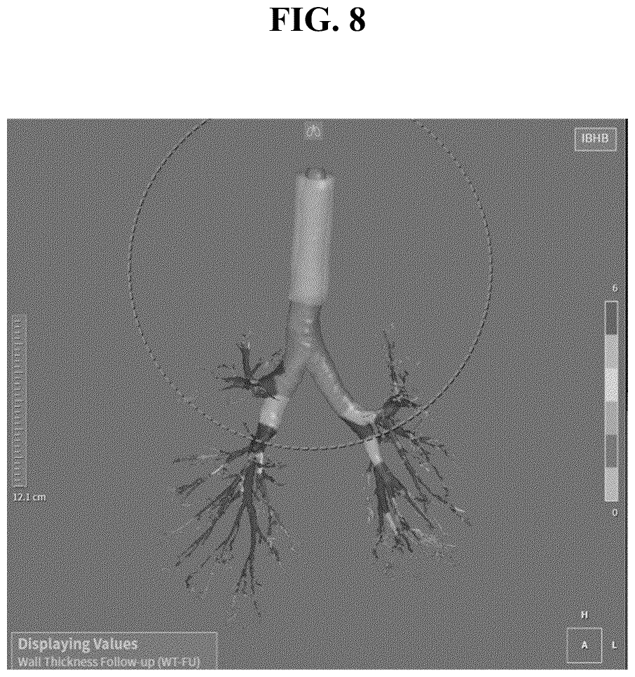

FIG. 8 is a conceptual diagram showing an embodiment of visualizing the follow-up quantitative information of an anatomical tubular structure according to an embodiment of the present invention.

Referring to FIG. 8, there is shown an embodiment of visualizing individual branch regions of the airway using different visual elements. In this case, each of the branch regions may be visualized using a visual element designated for each group as which the follow-up information of each branch region is classified. For example, in FIG. 8, the branch regions of a group having follow-up information whose quantitative information increases above a predetermined threshold may be visualized as the darkest regions.

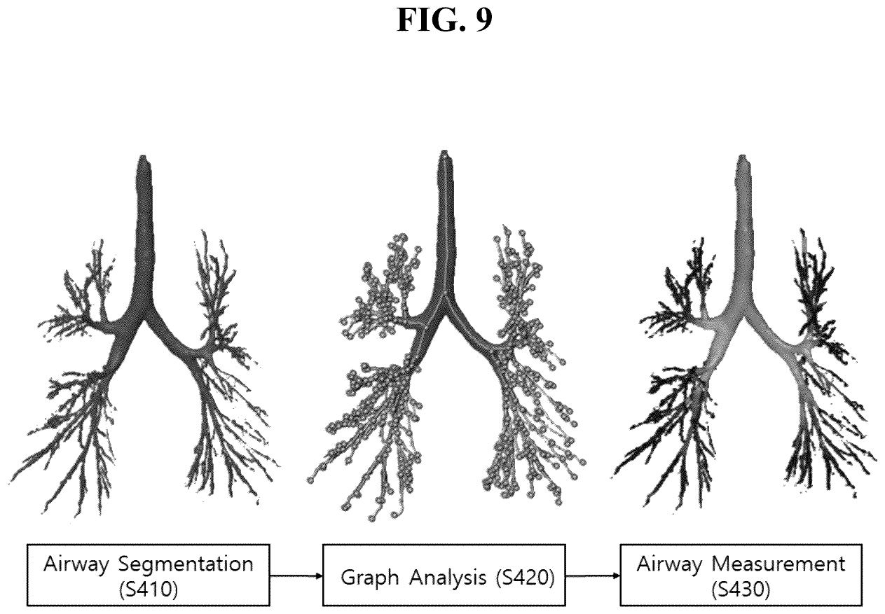

FIG. 9 is a conceptual diagram showing a process of generating the quantitative information of an anatomical tubular structure according to an embodiment of the present invention.

An anatomical tubular structure may be segmented for each of first medical image and second medical image in step S410.

The segmented anatomical tubular structure may be identified as branch regions corresponding to branched tree structures through graph analysis in step S420.

Quantitative information may be measured for the branch regions in step S430.

The process of FIG. 9 is performed for each of the first medical image and second medical image, and may be performed independently of the other process.

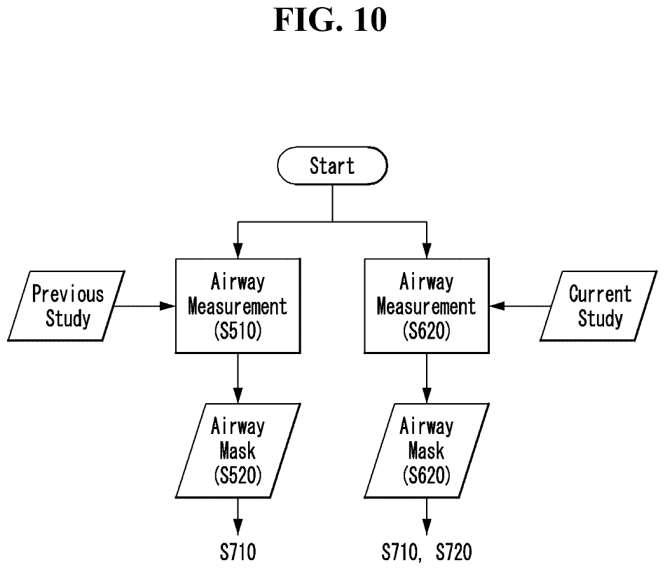

FIG. 10 is an operational flowchart showing part of a process of generating the follow-up quantitative information of an anatomical tubular structure according to an embodiment of the present invention.

Referring to FIG. 10, steps S410 to S430 for the first medical image obtained by the previous study are performed, and the quantitative information of each of the first branch regions in the airway area may be measured in step S510.

A mask may be generated for each of the first branch regions in the airway area in step S520, and quantitative information may be stored in association with each mask.

Steps S410 to S430 for the second medical image obtained by the current study may be performed, and the quantitative information of each of the second branch regions in the airway area may be measured in step S610.

A mask may be generated for each of the second branch regions in the airway area in step S620, and quantitative information may be stored in association with each mask.

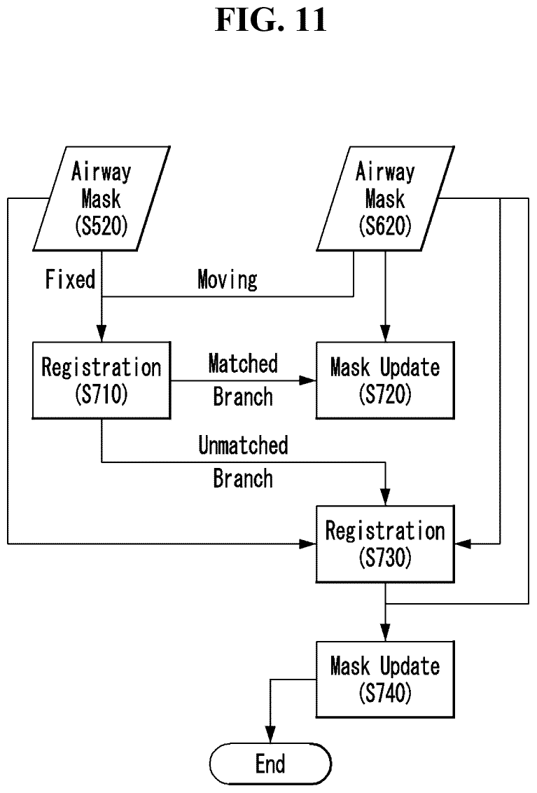

FIG. 11 is an operational flowchart showing another part of the process of generating the follow-up quantitative information of an anatomical tubular structure according to an embodiment of the present invention.

Referring to FIG. 11, in the state in which the mask for the first branch regions in the airway area is fixed, the mask for the second branch regions may be moved or deformed to perform registration between the branch regions in step S710.

The mask for the second branch regions may be updated for the branch regions matched by registration in step S720.

In this case, the quantitative information of the second branch regions may be updated, and follow-up information may be stored in association with the mask for the second branch regions with the quantitative information of the first branch region and the quantitative information of the second branch region reflected therein.

The registration S730 between unmatched branch regions may be performed again. The second registration S730 may use the same method as the first registration S710, or may use a different method.

In step S740, the mask for the second branch regions may be updated based on the follow-up information between branch regions that are additionally matched through the second registration S730.

In this case, although an embodiment in which the follow-up information is stored and updated in association with the mask for the second branch regions is shown, the first medical image may be selected as a reference image for follow-up analysis instead of the second medical image depending on the purpose of diagnosis according to an alternative embodiment of the present invention. In this case, the follow-up information may be updated in association with the mask for the first branch regions.

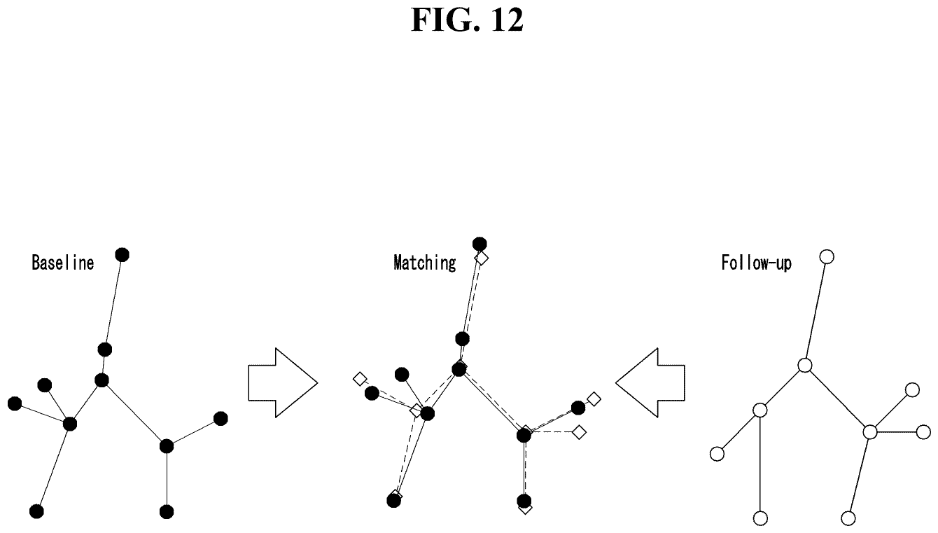

FIG. 12 is a conceptual diagram showing a process of the registration and matching of an anatomical tubular structure according to an embodiment of the present invention.

Referring to FIG. 12, there are shown the registration (S710) process and the matching process.

The information of branch regions identified in the first medical image (the baseline image) and the information of branch regions identified in the second medical image (the follow-up image) may be registered and matched.

In this case, the information of the first branch regions may be fixed, and the information of the second branch regions may be registered with the first branch regions and thus deformed or moved.

In this case, the registration between branch points may be performed based on the locations of the branch points and the levels of the branches before and after the branching of the branch points, and the registration between branches may be performed by considering similarity in branch level, location, size, and/or the like between the individual branches.

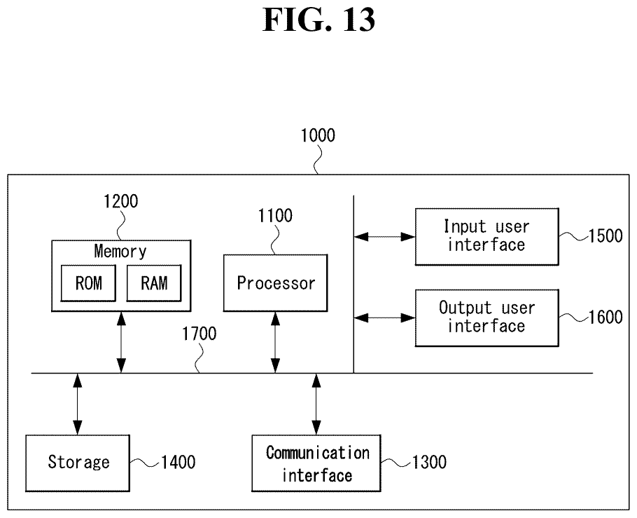

FIG. 13 is a conceptual diagram showing an example of an apparatus for generating and visualizing diagnostic assistant information for the follow-up analysis of a specific disease using generalized medical image processing, analysis and visualization, diagnosis assistance, and/or medical images, or an apparatus or computing system for assisting in the diagnosis of a specific disease through follow-up analysis that are capable of performing at least part of the processes of FIGS. 1 to 12.

At least part of a method of generating and visualizing diagnostic assistant information using medical image processing, analysis and visualization, diagnosis assistance, and/or medical images and the method of assisting in the diagnosis of a specific disease through follow-up analysis may be performed by the computing system 1000 of FIG. 13.

Referring to FIG. 13, the computing system 1000 according to an embodiment of the present invention may include a processor 1100, memory 1200, a communication interface 1300, storage 1400, an input user interface 1500, an output user interface 1600, and a bus 1700.

The computing system 1000 according to an embodiment of the present invention may include the at least one processor 1100, and the memory 1200 configured to store instructions causing the at least one processor 1100 to perform at least one step. At least part of a method according to an embodiment of the present invention may be performed in such a manner that the at least one processor 1100 loads at least one of the instructions from the memory 1200 and executes it.

The processor 1100 may refer to a central processing unit (CPU), a graphics processing unit (GPU), or a dedicated processor configured such that the methods according to the embodiments of the present invention are performed thereon.

Each of the memory 1200 and the storage 1400 may include at least one of a volatile storage medium and a non-volatile storage medium. For example, the memory 1200 may include at least one of read-only memory (ROM) and random access memory (RAM).

The computing system 1000 may further include the communication interface 1300 configured to perform communication over a wireless network.

The computing system 1000 may further include the storage 1400, the input user interface 1500, and the output user interface 1600.

The individual components included in the computing system 1000 may be connected through the bus 1700 and communicate with each other.

Examples of the computing system 1000 according to the present invention may include a communication-enabled desktop computer, laptop computer, notebook computer, smartphone, tablet personal computer (PC), mobile phone, smart watch, smart glass, e-book reader, portable multimedia player (PMP), portable game console, car navigation device, digital camera, digital multimedia broadcasting (DMB) player, digital audio recorder, digital audio player, digital video recorder, digital video player, and personal digital assistant (PDA), etc.

An apparatus for analyzing medical images to assist in diagnosing spinal disease according to an embodiment of the present invention may include memory 1200 configured to store one or more instructions, and a processor 1100 configured to load and execute at least one of the instructions from the memory.

In accordance with at least one of the instructions, the processor 1100 may obtain the first branch region of an anatomical tubular structure in a first medical image and the quantitative information of the first branch region in step S120, may identify a second branch region matching the first branch region of the anatomical tubular structure in a second medical image obtained after the first medical image in step S140, may obtain the quantitative information of the second branch region in step S160, and may generate diagnostic assistant information based on the quantitative information of the first branch region and the quantitative information of the second branch region in step S200.

When generating the diagnostic assistant information, the processor 1100 may generate follow-up information including the quantitative information of the first branch region and the quantitative information of the second branch region that match each other in step S220.

When generating the diagnostic assistant information, the processor 1100 may generate group correspondence information regarding which group is corresponding to at least one of the first branch region and/or the second branch region corresponding to the follow-up information from among a plurality of groups classified based on the quantitative information of the first branch region and the quantitative information of the second branch region included in the follow-up information in step S240.

When generating the group correspondence information in step S240, the processor 1100 may generate the group correspondence information by classifying each of the plurality of groups as at least one of a group in which the quantitative information of the second branch region has increased compared to the quantitative information of the first branch region, a group in which the quantitative information of the second branch region is the same as the quantitative information of the first branch region, and/or a group in which the quantitative information of the second branch region has decreased compared to the quantitative information of the first branch region.

When generating the group correspondence information in step S240, the processor 1100 may generate the group correspondence information by further classifying each of the plurality of groups as at least one of a group to which a new third branch region that does not match the first branch region corresponds among the branch regions found in the second medical image, and/or a group to which a fourth branch region that does not match the branch regions in the second medical image corresponds out of the first branch region.

In accordance with at least one of the instructions, the processor 1100 may visualize the diagnostic assistant information by visualizing the follow-up information together with the location of at least one of the first branch region and/or the second branch region corresponding to the follow-up information in steps S300 and S320.

In accordance with at least one of the instructions, the processor 1100 may visualize the diagnostic assistant information by visualizing group correspondence information regarding which group is corresponding to at least one of the first branch region and/or the second branch region corresponding to the follow-up information in steps S300 and S340.

When visualizing the diagnostic assistant information, the processor 1100 may visualize at least one of the first branch region and/or the second branch region using a visualization element designated for each group in steps S300 and S340.

According to an embodiment of the present invention, a consistent index for quantitative analysis may be provided using registration between the latest image and the previous image.

According to an embodiment of the present invention, there may be provided an assessment index, the visualization of the index, and assessment criteria for the quantitative assessment of the degree of progression of COPD or emphysema.

According to one embodiment of the present invention, an index capable of quantitatively assessing the degree of progression of a specific disease may be generated based on a change in quantitative information over time in a branch region by using follow-up matching between branch regions of an anatomical tubular structure in medical images.

In this case, criteria for determining the quantitative information of a branch region and/or criteria for assessing changes in the quantitative information of a branch region may be determined depending on a specific disease. Furthermore, a method for calculating the quantitative information of a branch region and/or changes in the quantitative information of a branch region may be selected from clinically known methods.

The operations of the method according to the exemplary embodiment of the present disclosure can be implemented as a computer readable program or code in a computer readable recording medium. The computer readable recording medium may include all kinds of recording apparatus for storing data which can be read by a computer system. Furthermore, the computer readable recording medium may store and execute programs or codes which can be distributed in computer systems connected through a network and read through computers in a distributed manner.

The computer readable recording medium may include a hardware apparatus which is specifically configured to store and execute a program command, such as a ROM, RAM or flash memory. The program command may include not only machine language codes created by a compiler, but also high-level language codes which can be executed by a computer using an interpreter.

Although some aspects of the present disclosure have been described in the context of the apparatus, the aspects may indicate the corresponding descriptions according to the method, and the blocks or apparatus may correspond to the steps of the method or the features of the steps. Similarly, the aspects described in the context of the method may be expressed as the features of the corresponding blocks or items or the corresponding apparatus. Some or all of the steps of the method may be executed by (or using) a hardware apparatus such as a microprocessor, a programmable computer or an electronic circuit. In some embodiments, one or more of the most important steps of the method may be executed by such an apparatus.

In some exemplary embodiments, a programmable logic device such as a field-programmable gate array may be used to perform some or all of functions of the methods described herein. In some exemplary embodiments, the field-programmable gate array may be operated with a microprocessor to perform one of the methods described herein. In general, the methods are preferably performed by a certain hardware device.

The description of the disclosure is merely exemplary in nature and, thus, variations that do not depart from the substance of the disclosure are intended to be within the scope of the disclosure. Such variations are not to be regarded as a departure from the spirit and scope of the disclosure. Thus, it will be understood by those of ordinary skill in the art that various changes in form and details may be made without departing from the spirit and scope as defined by the following claims.

What is claimed is:

1. A method of generating diagnostic assistant information using medical images, the method comprising:
obtaining a first branch region of an anatomical tubular structure in a first medical image and quantitative information of the first branch region;
identifying a second branch region matching the first branch region of the anatomical tubular structure in a second medical image obtained after the first medical image;
obtaining quantitative information of the second branch region; and
generating diagnostic assistant information based on the quantitative information of the first branch region and the quantitative information of the second branch region.

2. The method of claim 1, wherein the generating comprises generating follow-up information including the quantitative information of the first branch region and the quantitative information of the second branch region that match each other.

3. The method of claim 2, wherein the generating further comprises generating group correspondence information regarding which group is corresponding to at least one of the first branch region or the second branch region corresponding to the follow-up information from among a plurality of groups classified based on the quantitative information of the first branch region and the quantitative information of the second branch region included in the follow-up information.

4. The method of claim 3, wherein the generating group correspondence information comprises generating the group correspondence information by classifying each of the plurality of groups as at least one of a group in which the quantitative information of the second branch region has increased compared to the quantitative information of the first branch region, a group in which the quantitative information of the second branch region is the same as the quantitative information of the first branch region, or a group in which the quantitative information of the second branch region has decreased compared to the quantitative information of the first branch region.

5. The method of claim 4, wherein the generating group correspondence information comprises generating the group correspondence information by further classifying each of the plurality of groups as at least one of a group to which a new third branch region that does not match the first branch region corresponds among branch regions found in the second medical image, or a group to which a fourth branch region that does not match the branch regions in the second medical image corresponds out of the first branch region.

6. The method of claim 2, further comprising visualizing the diagnostic assistant information by visualizing the follow-up information together with a location of at least one of the first branch region or the second branch region corresponding to the follow-up information.

7. The method of claim 3, further comprising visualizing the diagnostic assistant information by visualizing group correspondence information regarding which group is corresponding to at least one of the first branch region or the second branch region corresponding to the follow-up information.

8. The method of claim 7, wherein the visualizing the diagnostic assistant information comprises visualizing at least one of the first branch region or the second branch region using a visualization element designated for each group.

9. The method of claim 1, wherein the quantitative information of at least one of the first branch region or the second branch region comprises at least one of a volume, diameter, or sectional area of the branch region.

10. The method of claim 1, wherein the quantitative information of at least one of the first branch region or the second branch region comprises at least one of a thickness of a wall of a tubular structure in the branch region, a sectional area of the wall of the tubular structure in the branch region, a diameter of a lumen of the tubular structure in the branch region, a sectional area of the lumen of the tubular structure in the branch region, a normalized wall thickness of the tubular structure in the branch region, or a tapering ratio of the tubular structure in the branch region.

11. The method of claim 3, wherein the generating the group correspondence information comprises generating the group correspondence information by using a plurality of groups for each of which a change between the quantitative information of the first branch region and the quantitative information of the second branch region included in the follow-up information is classified by applying a predetermined threshold.

12. The method of claim 1, wherein the anatomical tubular structure is at least one of an airway, an aorta, or a blood vessel.

13. An apparatus for generating diagnostic assistant information using medical images, the apparatus comprising:
memory configured to store one or more instructions; and
a processor configured to execute at least one of the instructions;
wherein the processor is further configured to, in accordance with at least one of the instructions:
obtain a first branch region of an anatomical tubular structure in a first medical image and quantitative information of the first branch region;
identify a second branch region matching the first branch region of the anatomical tubular structure in a second medical image obtained after the first medical image;
obtain quantitative information of the second branch region; and
generate diagnostic assistant information based on the quantitative information of the first branch region and the quantitative information of the second branch region.

14. The apparatus of claim 13, wherein the processor is further configured to, when generating the diagnostic assistant information, generate follow-up information including the quantitative information of the first branch region and the quantitative information of the second branch region that match each other.

15. The apparatus of claim 14, wherein the processor is further configured to, when generating the diagnostic assistant information, generate group correspondence information regarding which group is corresponding to at least one of the first branch region or the second branch region corresponding to the follow-up information from among a plurality of groups classified based on the quantitative information of the first branch region and the quantitative information of the second branch region included in the follow-up information.

16. The apparatus of claim 15, wherein the processor is further configured to, when generating the group correspondence information, generate the group correspondence information by classifying each of the plurality of groups as at least one of a group in which the quantitative information of the second branch region has increased compared to the quantitative information of the first branch region, a group in which the quantitative information of the second branch region is the same as the quantitative information of the first branch region, or a group in which the quantitative information of the second branch region has decreased compared to the quantitative information of the first branch region.

17. The apparatus of claim 16, wherein the processor is further configured to, when generating the group correspondence information, generate the group correspondence information by further classifying each of the plurality of groups as at least one of a group to which a new third branch region that does not match the first branch region corresponds among branch regions found in the second medical image, or a group to which a fourth branch region that does not match the branch regions in the second medical image corresponds out of the first branch region.

18. The apparatus of claim 14, wherein the processor is further configured to, in accordance with at least one of the instructions, visualize the diagnostic assistant information by visualizing the follow-up information together with a location of at least one of the first branch region or the second branch region corresponding to the follow-up information.

19. The apparatus of claim 15, wherein the processor is further configured to, in accordance with at least one of the instructions, visualize the diagnostic assistant information by visualizing group correspondence information regarding which group is corresponding to at least one of the first branch region or the second branch region corresponding to the follow-up information.

20. The apparatus of claim 19, wherein the processor is further configured to, when visualizing the diagnostic assistant information, visualize at least one of the first branch region or the second branch region using a visualization element designated for each group.
